(19) United States
(12) Patent Application Publication
(10) Pub. No.: US 2020/0096427 A1
(43) Pub. Date: Mar. 26, 2020

(54) GREASE PROPERTY MEASUREMENT DEVICE AND GREASE PROPERTY MEASUREMENT METHOD

(71) Applicant: JTEKT CORPORATION, Osaka (JP)

(72) Inventor: Hirotaka YASUDA, Kashihara-shi (JP)

(73) Assignee: JTEKT CORPORATION, Osaka (JP)

(21) Appl. No.: 16/553,523

(22) Filed: Aug. 28, 2019

(30) Foreign Application Priority Data
Sep. 25, 2018 (JP) 2018-178231

(51) Int. Cl. G01N 11/04; G01N 33/28; G01N 33/30

(52) U.S. Cl. CPC G01N 11/04; G01N 33/30; G01N 33/2888

(57) ABSTRACT

A grease property measurement device includes a flow pipe having a first end that communicates with an inside of a rolling bearing such that grease discharged from the rolling bearing flows through the flow pipe; a reservoir unit including an inlet port to which a second end of the flow pipe is connected such that the grease is introduced from the flow pipe through the inlet port, a reservoir chamber which stores the grease introduced through the inlet port, and a discharge port through which the grease is discharged from the reservoir chamber; an extrusion mechanism configured to push the grease in the reservoir chamber so as to discharge the grease through the discharge port; and a measuring unit configured to measure flow resistance at a time when the extrusion mechanism pushes the grease in the reservoir chamber.

### INCORPORATION BY REFERENCE

The disclosure of Japanese Patent Application No. 2018-178231 filed on Sep. 25, 2018 including the specification, drawings and abstract is incorporated herein by reference in its entirety.

### BACKGROUND

### 1. Technical Field

The disclosure relates to a grease property measurement device and a grease property measurement method that can be applied to a rolling bearing configured to support a conveyor roller, a rolling bearing configured to support a main shaft of a wind power generation system, and the like.

### 2. Description of Related Art

For example, in general, in a wind power generation system, a blade connected to a main shaft receives wind to rotate the main shaft, the rotation of the main shaft is transmitted to a generator, and thus power is generated. The main shaft of this wind power generation system is supported so as to be rotatable by the rolling bearing. In addition, an axial load or a radial load is applied to the main shaft by the wind received by the blade, and the main shaft is thereby bent during operation. Accordingly, a self-aligning roller bearing capable of absorbing the bending of the main shaft is mainly used for the rolling bearing.

The rolling bearing, which supports the main shaft, is lubricated with grease filled therein. However, because the grease is deteriorated by use, the grease needs to be replaced according to a degree of the deterioration. Conventionally, the grease in the rolling bearing is partially removed during a periodic inspection of the wind power generation system, and the degree of the deterioration of the grease is checked by a person on the basis of consistency (cone penetration) and the like of the grease. As a result, in the case where it is determined that the deterioration of the grease has progressed, the grease is replaced.

For example, Japanese Patent Application Publication No. 2012-154472 (JP 2012-154472 A) describes a maintenance device for a wind power generation system, which includes a supply pump that automatically supplies the grease; and a pressure sensor that measures a supply pressure of the grease. In the technique described in JP 2012-154472 A, the supply pressure of the grease that changes according to the degree of the deterioration of the grease in the rolling bearing is periodically and automatically measured and the degree of the deterioration of the grease is determined based on the supply pressure.

### SUMMARY

With the method for checking the consistency or the like of the grease in the rolling bearing by a person during the periodic inspection of the wind power generation system, the grease can be replaced only at the time of the periodic inspection. However, the timing of the periodic inspection does not always coincide with timing at which the grease should be replaced, for example, in a case where the grease is deteriorated early. Thus, with the above method, there is a possibility that the grease cannot be replaced at the appropriate timing. Furthermore, for the above method, a worker needs to visit a nacelle, which is located at a high position, in the wind power generation system to check the consistency of the grease. As a result, work becomes bothersome.

Meanwhile, the technique described in JP 2012-154472 A is based on the premise that the grease is automatically supplied, and thus the technique cannot be applied to the wind power generation system that does not have a facility for the automatic supply. In addition, in the technique, the degree of the deterioration of the used grease is indirectly determined based on the pressure at the time when new grease is supplied into the rolling bearing. Thus, the determination is influenced by the newly supplied grease. Furthermore, in the case where the rolling bearing or a housing has an internal space where the grease does not exist, for example, in the case where the space is generated when the grease is pushed out of the rolling bearing or the housing by rotation of the rolling bearing or when the grease leaks out from a seal between the shaft and the housing, such a space has an influence on the pressure at the time when the new grease is supplied. Thus, with the technique described in JP 2012-154472 A, it is difficult to accurately determine the degree of the deterioration of the grease.

The disclosure provides a grease property measurement device and a grease property measurement method that can measure a property of used grease in a rolling bearing at a location away from the rolling bearing.

A first aspect of the disclosure relates to a grease property measurement device including a flow pipe having a first end that communicates with an inside of a rolling bearing such that grease discharged from the rolling bearing flows through the flow pipe; a reservoir unit including an inlet port to which a second end of the flow pipe is connected such that the grease is introduced from the flow pipe through the inlet port, a reservoir chamber which stores the grease introduced through the inlet port, and a discharge port through which the grease is discharged from the reservoir chamber; an extrusion mechanism configured to push the grease in the reservoir chamber so as to discharge the grease through the discharge port; and a measuring unit configured to measure flow resistance at a time when the extrusion mechanism pushes the grease in the reservoir chamber.

In the property measurement device with the above-described configuration, the grease in the rolling bearing is introduced through the flow pipe and is stored in the reservoir chamber of the reservoir unit. The grease in the reservoir chamber is pushed by the extrusion mechanism and is discharged through the discharge port. The measuring unit measures the flow resistance of the grease at this time. The flow resistance measured by the measuring unit is correlated with consistency (cone penetration) that is one of the properties of the grease. Thus, the consistency of the grease can be calculated from the flow resistance. In addition, because consistency of the grease is changed due to deterioration, a degree of the deterioration of the grease can be determined on the basis of the consistency. Thus, the grease can be replaced at appropriate timing. Furthermore, the grease in the rolling bearing is introduced into the reservoir chamber of the reservoir unit via the flow pipe. Accordingly, the property of the grease can be measured at a location away from the rolling bearing.

The extrusion mechanism may include a piston provided to reciprocate in a first direction to push the grease in the reservoir chamber and in a second direction opposite to the first direction; and a drive unit configured to cause the piston to reciprocate. With the configuration, the grease in the reservoir chamber can be pushed by the piston, and can be discharged through the discharge port. Thereafter, the piston can be returned to an original position.

The measuring unit may include a pressure sensor that is provided between the piston and the drive unit, and the pressure sensor may detect a pressure applied to the piston from the drive unit. In the case where the flow resistance of the grease that is discharged from the reservoir chamber is high, a pressure that is applied from the drive unit to the piston is increased. To the contrary, in the case where the flow resistance is low, the pressure that is applied from the drive unit to the piston is reduced. Thus, the flow resistance of the grease can be measured based on the pressure that is detected by the pressure sensor.

The extrusion mechanism may further include a coupling unit that couples the piston and the drive unit; and the coupling unit may be configured to cause the pressure sensor to contact both of the drive unit and the piston when the piston moves in the first direction, and to cause the pressure sensor to move away from one of the drive unit and the piston when the piston moves in the second direction. With this configuration, the pressure sensor can detect the pressure only at the time when the piston moves in the first direction to push out the grease.

The grease property measurement device may further include a check valve configured to permit a flow of the grease in a direction from the flow pipe toward the inlet port and to prevent a flow of the grease in a direction opposite to the direction from the flow pipe toward the inlet port. With this configuration, when the extrusion mechanism pushes the grease in the reservoir chamber, it is possible to prevent a reverse flow of the grease from the inlet port to the flow pipe.

A second aspect of the disclosure relates to a grease property measurement method including introducing grease in a rolling bearing into a reservoir chamber through a flow pipe; pushing the grease in the reservoir chamber so as to discharge the grease from the reservoir chamber; and measuring flow resistance at a time when the grease is pushed and discharged from the reservoir chamber.

In the property measurement method with the above-described configuration, the grease in the rolling bearing is introduced through the flow pipe and is stored in the reservoir chamber, the grease in the reservoir chamber is pushed and discharged from the reservoir chamber through the discharge port by the extrusion mechanism, and the flow resistance at the time of pushing the grease is measured. The flow resistance is correlated with the consistency (the cone penetration) that is one of the properties of the grease. Thus, the consistency of the grease can be calculated from the flow resistance. In addition, because the consistency of the grease is changed due to the deterioration, the degree of the deterioration of the grease can be determined on the basis of the measured flow resistance. Thus, the grease can be replaced at appropriate timing. Furthermore, because the grease in the rolling bearing is introduced into the reservoir chamber through the flow pipe, the property of the grease can be measured at the location away from the rolling bearing.

The grease property measurement device and the grease property measurement method according to the above aspects of the disclosure can directly detect the property of the used grease in the rolling bearing at the location away from the rolling bearing.

### BRIEF DESCRIPTION OF THE DRAWINGS

Features, advantages, and technical and industrial significance of exemplary embodiments of the disclosure will be described below with reference to the accompanying drawings, in which like numerals denote like elements, and wherein:

FIG. 1 is a schematic side view of a grease property measurement device according to an embodiment;

FIG. 2 is a sectional view of a reservoir unit in the property measurement device;

FIG. 3 is a perspective view of a coupling portion between a drive unit and a piston in the property measurement device;

FIG. 4 is a side view of the coupling portion between the drive unit and the piston in the property measurement device;

FIGS. 5A, 5B are side views for illustrating operation in the coupling portion between the drive unit and the piston;

FIGS. 6A, 6B, 6C are sectional views for illustrating operation of the property measurement device; and

FIGS. 7A, 7B are sectional views of modified examples of the reservoir unit in the property measurement device.

### DETAILED DESCRIPTION OF EMBODIMENTS

A detailed description will hereinafter be made on an embodiment of the disclosure with reference to the accompanying drawings. The disclosure is not limited to embodiment described below. Various modifications may be made to the embodiment within the scope of the disclosure.

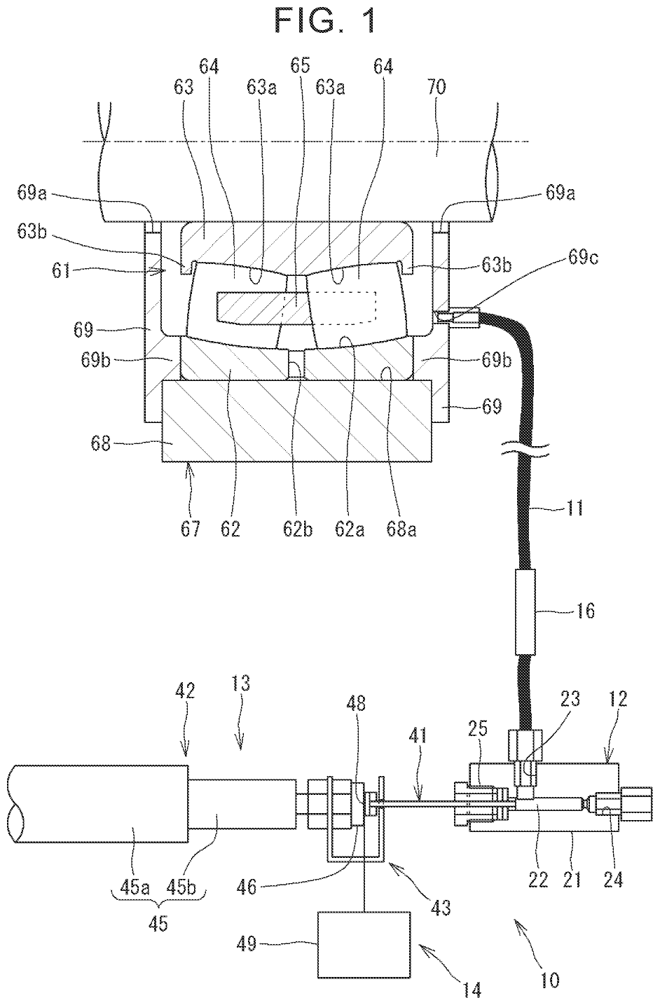

FIG. 1 is a schematic side view of a grease property measurement device according to an embodiment. A property measurement device 10 according to this embodiment is a device that measures a property of grease filled in a rolling bearing 61. The rolling bearing 61 as a measurement target in this embodiment is a rolling bearing that supports a main shaft 70 of the wind power generation system such that the main shaft 70 is rotatable, for example. In general, as this rolling bearing 61, a self-aligning roller bearing to which a radial load and an axial load can be applied and which can absorb bending of the main shaft 70 is adopted. The rolling bearing 61 is accommodated in a bearing housing 67.

A configuration of the rolling bearing 61 will be described. The rolling bearing 61 includes an outer ring 62, an inner ring 63, rolling elements 64, and a cage 65. The outer ring 62 has a ring shape. A raceway surface 62a in a concave spherical surface shape is formed on an inner periphery of the outer ring 62. A grease injection hole 62b is formed in a central portion of the outer ring 62 in an axial direction. The grease is supplied into the bearing housing 67 from a grease supply port (not shown) formed in the bearing housing 67, and is then filled into the rolling bearing 61 from the grease injection hole 62b.

The inner ring 63 has a ring shape. A plurality of rows of raceway surfaces 63a in curved surface shapes are formed on an outer periphery of the inner ring 63 such that a center of the outer periphery in the axial direction is projected. A pair of flanges 63b is provided, that is, the flanges 63b are respectively provided at both ends of the outer periphery of the inner ring 63 in the axial direction. The main shaft 70 is press-fitted into an inner peripheral surface of the inner ring 63, and the inner ring 63 is fixed to the main shaft 70 such that the inner ring 63 is rotatable integrally with the main shaft 70.

The rolling elements 64 are spherical rollers that are arranged so as to be rollable in a plurality of rows between the raceway surface 62a of the outer ring 62 and the raceway surfaces 63a of the inner ring 63. Outward movement of each of the rolling elements 64 in the axial direction is restricted by the pair of flanges 63b, and thus the rolling elements 64 are prevented from falling out of the rolling bearing 61. The rolling bearing 61 can absorb deformation caused by, for example, the bending of the main shaft 70 when the rolling elements 64 move in the axial direction on the raceway surface 62a of the outer ring 62.

The bearing housing 67 includes a housing body 68 and lid bodies 69. The housing body 68 is provided with a mounting hole 68a to which the outer ring 62 is fitted. An outer peripheral surface of the outer ring 62 is fitted to the mounting hole 68a. The lid bodies 69 cover a ring-shaped space between the mounting hole 68a of the housing body 68 and the main shaft 70 from both sides in the axial direction. In a central portion of the lid body 69 in a disc shape, an opening 69a, through which the main shaft 70 passes, is formed. The lid body 69 is fixed to a side surface of the housing body 68 in the axial direction by a bolt or the like. One side surface of the lid body 69 located on the side of the rolling bearing 61 is provided with a ring-shaped projection 69b that is projected in a direction toward the outer ring 62 and is fitted to the mounting hole 68a of the housing body 68.

The grease is filled (supplied) into a ring-shaped space between the outer ring 62 and the inner ring 63 of the rolling bearing 61. Leakage of the grease to the outside is prevented by the lid body 69. The lid body 69 is provided with a discharge hole 69c, from which the grease filled in the rolling bearing 61 is discharged to the outside. This discharge hole 69c is used to supply the grease filled in the rolling bearing 61 to the property measurement device 10.

The configuration of the property measurement device 10 will be described. The property measurement device 10 includes a flow pipe 11, a reservoir unit 12, an extrusion mechanism 13, and a measuring unit 14. The flow pipe 11 is a pipe through which the grease can flow. A first end of the flow pipe 11 is connected to the discharge hole 69c that is formed in the lid body 69 of the bearing housing 67. In this way, the first end of the flow pipe 11 communicates with the inside of the rolling bearing 61. The flow pipe 11 allows a flow of the grease that is discharged from the inside of the rolling bearing 61 via the discharge hole 69c. The flow pipe 11 is provided with a check valve 16. This check valve 16 permits the flow of the grease in a direction in which the grease is discharged from the rolling bearing 61 while preventing the flow of the grease in a reverse direction (i.e., an opposite direction).

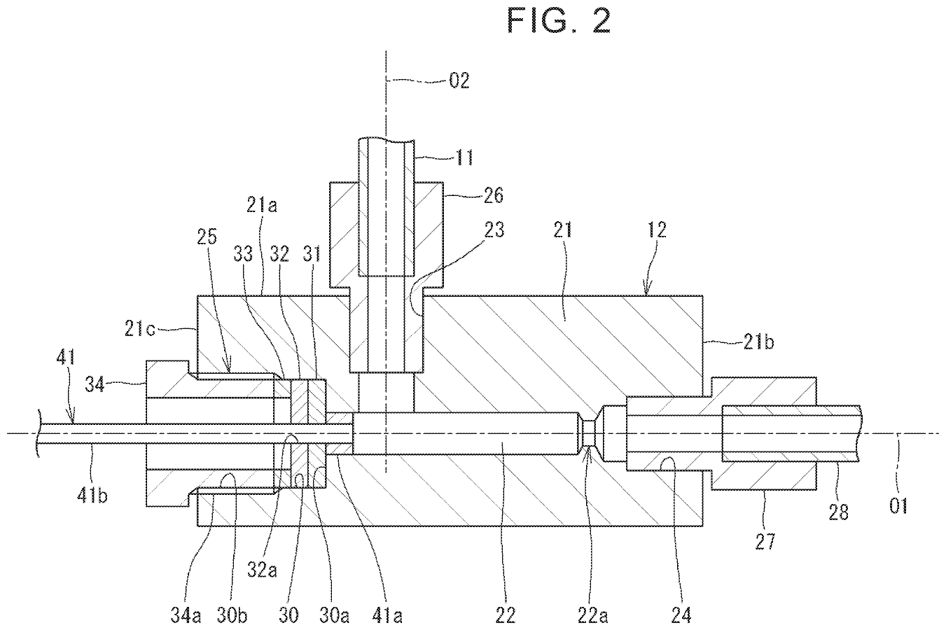

FIG. 2 is a sectional view of the reservoir unit 12 in the property measurement device 10. The reservoir unit 12 is configured to store the grease for a purpose of measuring the property of the grease. The reservoir unit 12 includes a body portion 21 in a substantially rectangular parallelepiped shape that is formed of metal, a hard resin, or the like. In the body portion 21, a reservoir chamber 22, an inlet port 23, a discharge port 24, and a piston support portion 25 are provided.

The reservoir chamber 22 is a space in which the grease is stored, and is formed in the body portion 21. The reservoir chamber 22 is a cylindrical hole that is formed along a longitudinal direction of the body portion 21. In the reservoir chamber 22, a piston head 41a of the extrusion mechanism 13, which will be described later, is accommodated to be movable along a length direction (a cylinder axis direction) of the reservoir chamber 22. In addition, a throttle portion 22a is formed at a second end portion (a right end portion in FIG. 2) of the reservoir chamber 22 in the length direction. The throttle portion 22a sharply reduces a cross-sectional area of the reservoir chamber 22.

The inlet port 23 is an opening through which the grease is introduced into the reservoir chamber 22 from the outside of the body portion 21. The inlet port 23 is a cylindrical hole that extends from one side surface 21a of the body portion 21 to a peripheral surface at a first end portion (a left end portion in FIG. 2) of the reservoir chamber 22 in the length direction. A center line (a cylindrical axis) O2 of the inlet port 23 is perpendicular (orthogonal) to a center line (the cylindrical axis) O1 of the reservoir chamber 22. A joint 26 is attached to the inlet port 23, and a second end of the flow pipe 11 is connected to the joint 26. Thus, the grease that flows through the flow pipe 11 is introduced into the reservoir chamber 22 from the inlet port 23.

The discharge port 24 is an opening through which the grease stored in the reservoir chamber 22 is discharged to the outside of the body portion 21. The discharge port 24 is a cylindrical hole that is formed between one end surface 21b of the body portion 21 in the length direction and the second end portion of the reservoir chamber 22 in the length direction. A center line (a cylindrical axis) of the discharge port 24 matches the center line O1 of the reservoir chamber 22, and the discharge port 24 and the reservoir chamber 22 are formed on a straight line. A discharge pipe 28 is connected to this discharge port 24 via a joint 27.

The piston support portion 25 supports a piston rod 41b of the extrusion mechanism 13, which will be described later. The piston support portion 25 includes an attachment hole 30, a seal member 31, a support ring 32, a spacer 33, and a fixing member 34.

The attachment hole 30 is a cylindrical hole that is formed between the other end surface 21c of the body portion 21 in the length direction and the other end of the reservoir chamber 22 in the length direction. A center line of the attachment hole 30 matches the center line O1 of the reservoir chamber 22, and both of them are arranged on the straight line. An inside diameter of the attachment hole 30 is larger than an inside diameter of the reservoir chamber 22. Accordingly, a step surface 30a is formed on a boundary between the attachment hole 30 and the reservoir chamber 22 due to a difference of the inside diameters thereof.

In the attachment hole 30, the seal member 31, the support ring 32, and the spacer 33 are accommodated in this order from the step surface 30a-side. The seal member 31 is formed of an elastic material such as rubber. The seal member 31 is formed in a ring shape having an outside diameter that is substantially the same as or slightly smaller than the inside diameter of the attachment hole 30. The seal member 31 has such a dimension that an inside diameter thereof is slightly larger than an outside diameter of the piston rod 41b in the extrusion mechanism 13. The seal member 31 prevents a flow of air between the reservoir chamber 22 and the attachment hole 30 because the piston head 41a of the extrusion mechanism 13 is tightly attached to the seal member 31.

The support ring 32 is formed of metal or a synthetic resin. The support ring 32 is formed in a ring shape having an outside diameter that is substantially the same as or slightly smaller than the inside diameter of the attachment hole 30. The support ring 32 has such a dimension that an inside diameter thereof is slightly larger than the outside diameter of the piston rod 41b in the extrusion mechanism 13. This support ring 32 supports the piston rod 41b such that the piston rod 41b is slidable.

The spacer 33 keeps a distance between the fixing member 34 and the support ring 32. The spacer 33 is formed in a cylindrical shape having a slightly smaller outside diameter than the inside diameter of the attachment hole 30. The fixing member 34 fixes the seal member 31, the support ring 32, and the spacer 33, which are accommodated in the attachment hole 30, in the attachment hole 30. The fixing member 34 is formed in a substantially cylindrical shape, and a male thread 34a is formed in a part of an outer peripheral surface thereof. The male thread 34a of the fixing member 34 is fastened to a female thread 30b that is formed in a part of an inner peripheral surface of the attachment hole 30.

As shown in FIG. 1, the extrusion mechanism 13 includes the piston 41, a drive unit 42, and a coupling unit 43. As shown in FIG. 2, the piston 41 includes the piston head 41a and the piston rod 41b.

The piston head 41a is formed in a columnar shape and is accommodated so as to be slidable in the reservoir chamber 22. The piston rod 41b is a rod body in a columnar shape. The piston rod 41b is slidably inserted in a center hole 32a of the support ring 32. The piston head 41a is fixed to one end of the piston rod 41b in the length direction. The piston rod 41b has the smaller outside diameter than the piston head 41a.

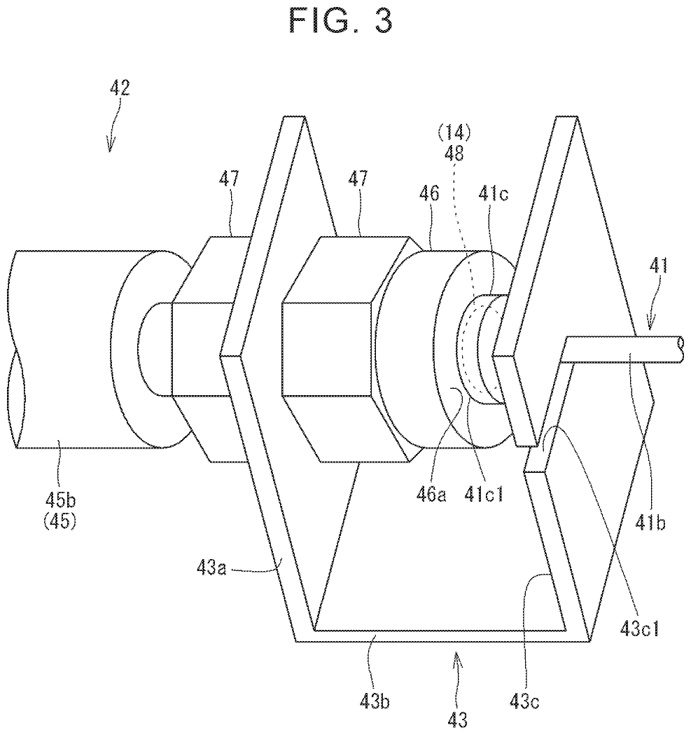

As shown in FIG. 3, the piston 41 further includes a load receiving member 41c that is provided at the other end of the piston rod 41b in the length direction. This load receiving member 41c is formed in a disc shape and receives a load from the drive unit 42.

As shown in FIG. 1 and FIG. 2, the drive unit 42 drives the piston 41 and causes the piston head 41a of the piston 41 to reciprocate in the reservoir chamber 22. The drive unit 42 includes a drive actuator 45 and a pressing member 46. For example, the drive actuator 45 is a contractible/extensible cylinder such as a known electric cylinder having a ball screw mechanism therein, or a fluid pressure cylinder using a fluid pressure such as a hydraulic pressure. The drive actuator 45 includes a cylinder body 45a; and a piston member 45b that is provided to be movable in the length direction in the cylinder body 45a.

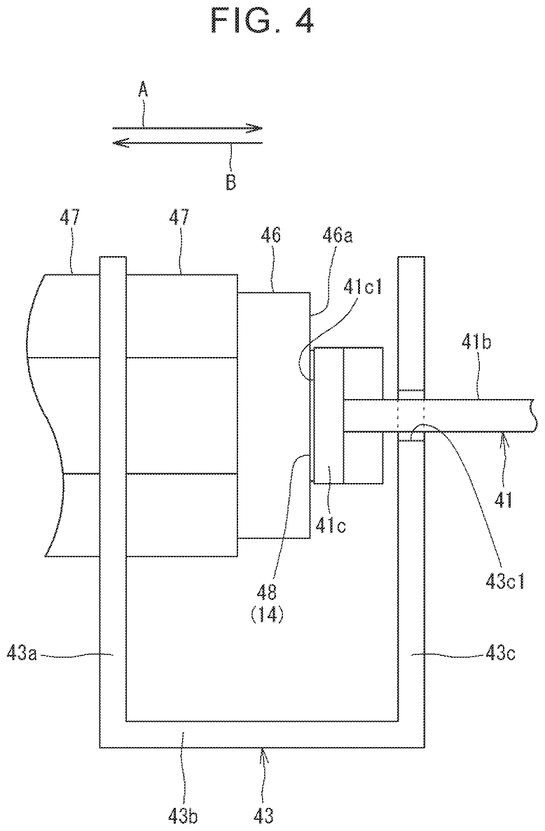

FIG. 3 is a perspective view of a coupling portion between the drive unit 42 and the piston 41 in the property measurement device 10, and FIG. 4 is a side view of the same. The pressing member 46 is attached to a distal end of the piston member 45b. More specifically, two nuts 47 are fastened in alignment to the distal end of the piston member 45b, and the pressing member 46 is attached to the nut 47 closer to the distal end of the extrusion mechanism 13. The pressing member 46 is formed in a disc shape. The pressing member 46 is disposed such that one side surface (a pressing surface) 46a thereof faces one side surface (a load receiving surface) 41c1 of the load receiving member 41c.

The coupling unit 43 couples the drive unit 42 and the piston 41. More specifically, the coupling unit 43 includes an attachment plate 43a, a coupling plate 43b, and a locking plate 43c. Each of the plates is formed in a rectangular shape. In addition, the coupling unit 43 is formed in a substantially U-shape, the attachment plate 43a and the locking plate 43c are arranged to face each other, and the attachment plate 43a and the locking plate 43c are coupled to each other by the coupling plate 43b.

The attachment plate 43a is attached to the distal end of the piston member 45b in the drive actuator 45. More specifically, at the distal end of the piston member 45b, the attachment plate 43a is attached and fixed between the two nuts 47. The locking plate 43c is provided with a cut groove 43c1, and the piston rod 41b of the piston 41 is inserted in this cut groove 43c1.

When the drive actuator 45 of the drive unit 42 is extended, the pressing member 46 presses the load receiving member 41c of the piston 41 in an arrow A direction (a first direction) indicated in FIG. 4. Thus, in the reservoir chamber 22 of the reservoir unit 12, the piston head 41a of the piston 41 moves from the first end portion (the left end portion in FIG. 2) to the second end portion (the right end portion in FIG. 2) of the reservoir chamber 22. In the case where the grease is stored in the reservoir chamber 22, the grease in the reservoir chamber 22 is discharged through the discharge port 24 due to the movement of this piston head 41a.

To the contrary, when the drive actuator 45 is contracted, the piston 41 is pulled in an arrow B direction (a second direction) indicated in FIG. 4 via the coupling unit 43. More specifically, the load receiving member 41c is locked to the locking plate 43c of the coupling unit 43, and the piston 41 is pulled in the arrow B direction. Thus, in the reservoir chamber 22, the piston head 41a moves from the second end portion to the first end portion of the reservoir chamber 22. By the operation described so far, the piston head 41a of the piston 41 reciprocates in the reservoir chamber 22.

The measuring unit 14 measures flow resistance of the grease at the time when the grease in the reservoir chamber 22 is pushed and discharged. More specifically, the measuring unit 14 detects a pressure that is applied from the drive actuator 45 of the drive unit 42 to the piston 41, and then measures the flow resistance of the grease based on the pressure. The measuring unit 14 includes a pressure sensor (a pressure-sensitive sensor) 48 and a detection circuit 49 (see FIG. 1).

The pressure sensor 48 is disposed between the pressing surface 46a of the pressing member 46 and the load receiving surface 41c1 of the load receiving member 41c and can contact both of the surfaces 46a, 41c1. The electric resistance of the pressure sensor 48 is changed when the pressure is applied to the pressures sensor 48. In addition, the pressure sensor 48 is attached to one of the pressing surface 46a and the load receiving surface 41c1. The pressure sensor 48 in this embodiment is attached to the pressing surface 46a. Note that the pressing surface 46a and the load receiving surface 41c1 are arranged in parallel with each other.

The detection circuit 49 is an electric circuit that outputs a voltage value applied to the pressure sensor 48 as a detection signal. This voltage value changes due to the change in the resistance value of the pressure sensor 48. Thus, the pressure that is applied to the pressure sensor 48 can be calculated based on the voltage value. In addition, in the case where consistency (cone penetration) of the grease in the reservoir chamber 22 is high, the flow resistance of the grease is increased. As a result, the pressure that is applied to the piston 41 from the drive actuator 45 is increased. To the contrary, in the case where the consistency (cone penetration) of the grease in the reservoir chamber 22 is low, the pressure that is applied to the piston 41 from the drive actuator 45 is reduced. Thus, the flow resistance of the grease can be calculated from the pressure that is applied to the pressure sensor 48.

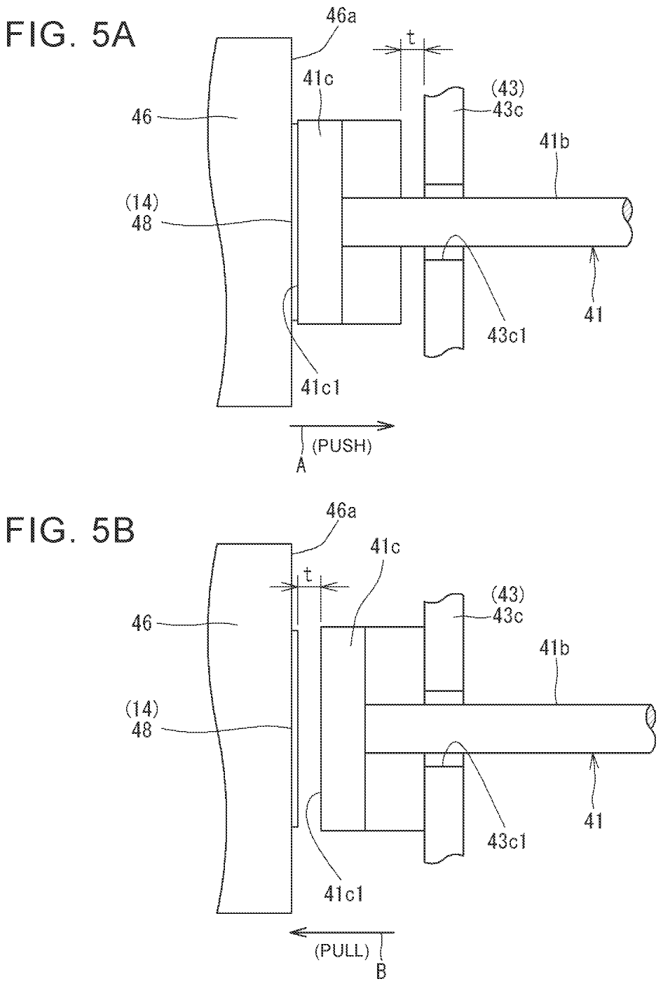

As shown in FIG. 5A, when the pressing member 46 presses the load receiving member 41c of the piston 41, the pressure sensor 48 is sandwiched between the pressing surface 46a of the pressing member 46 and the load receiving surface 41c1 of the load receiving member 41c. As a result, the pressure sensor 48 can measure the pressure that is applied from the pressing member 46 to the load receiving member 41c. At this time, a clearance t is generated between the load receiving member 41c and the locking plate 43c of the coupling unit 43.

As shown in FIG. 5B, when the drive actuator 45 pulls the piston 41 via the coupling unit 43, the load receiving member 41c moves away from the measuring unit 14 attached to the pressing member 46 while the clearance t is generated between the load receiving member 41c and the measuring unit 14. As a result, the load is not applied to the measuring unit 14, and the pressure is not detected. Thus, the coupling unit 43 is configured such that the pressure sensor 48 can detect the pressure only when the grease in the reservoir chamber 22 is discharged.

Note that, in the case where the pressing surface 46a of the pressing member 46 and the load receiving surface 41c1 of the load receiving member 41c are not arranged in parallel with each other, there is a possibility that the pressure is not applied to the pressure sensor 48 evenly (uniformly) and thus an appropriate measurement cannot be made. For such a reason, at least one of the pressing member 46 and the load receiving member 41c may be attached to the drive actuator 45 and/or the piston rod 41b via an elastic material such as the rubber. With this configuration, an inclination of one of the pressing surface 46a and the load receiving surface 41c1 with respect to the other can be elastically absorbed. Alternatively, the inclination can be also absorbed mechanically via a spherical joint or the like.

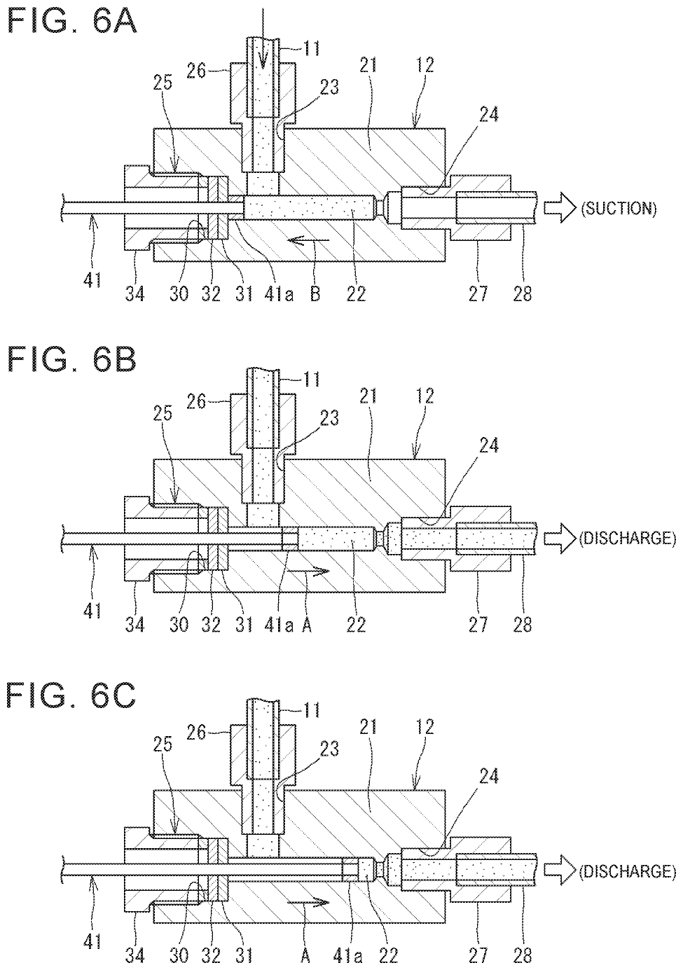

A description will hereinafter be made on operation of the property measurement device 10. FIGS. 6A, 6B, 6C are sectional views for illustrating the operation of the property measurement device 10. As shown in FIG. 6A, first, the grease in the rolling bearing 61 is introduced into the reservoir chamber 22 of the reservoir unit 12 and is stored therein. More specifically, a suction device (not shown) such as a pump is connected to the discharge pipe 28, which is connected to the discharge port 24 of the reservoir unit 12, and a negative pressure is generated in the reservoir chamber 22 by this suction device. Thus, the grease in the rolling bearing 61 is suctioned into the reservoir chamber 22 via the flow pipe 11. At this time, the piston head 41a of the piston 41 is pulled in the arrow B direction by the drive actuator 45, and is tightly attached to the seal member 31 in the piston support portion 25. Accordingly, leakage of air between the reservoir chamber 22 and the attachment hole 30 of the piston support portion 25 can be prevented.

Next, as shown in FIGS. 6B, 6C, when the drive actuator 45 is operated, the grease in the reservoir chamber 22 is pushed by the piston 41 and is discharged through the discharge port 24. The check valve 16 (see FIG. 1) is provided in the flow pipe 11. Thus, at this time, a reverse flow of the grease toward the rolling bearing 61 via the flow pipe 11 is prevented until the piston head 41a moves to a position shown in FIG. 6B.

In addition, the throttle portion 22a is provided at the end of the reservoir chamber 22, the end being close to the discharge port 24. Thus, the flow resistance of the grease at the time of pushing the grease in the reservoir chamber 22 is increased. For this reason, the pressure sensor 48 can reliably detect the pressure that is applied to the piston 41 from the drive actuator 45.

In this embodiment, the grease in the rolling bearing 61 is delivered into the reservoir unit 12 via the flow pipe 11. Accordingly, the property of the grease can be measured at a location away from the rolling bearing 61. In addition, a degree of deterioration of the grease can be determined on the basis of a result of measurement of the property, and thus the grease can be replaced at appropriate timing. Furthermore, the property measurement device 10 in this embodiment can directly measure the property of the grease that is used and deteriorated in the rolling bearing 61.

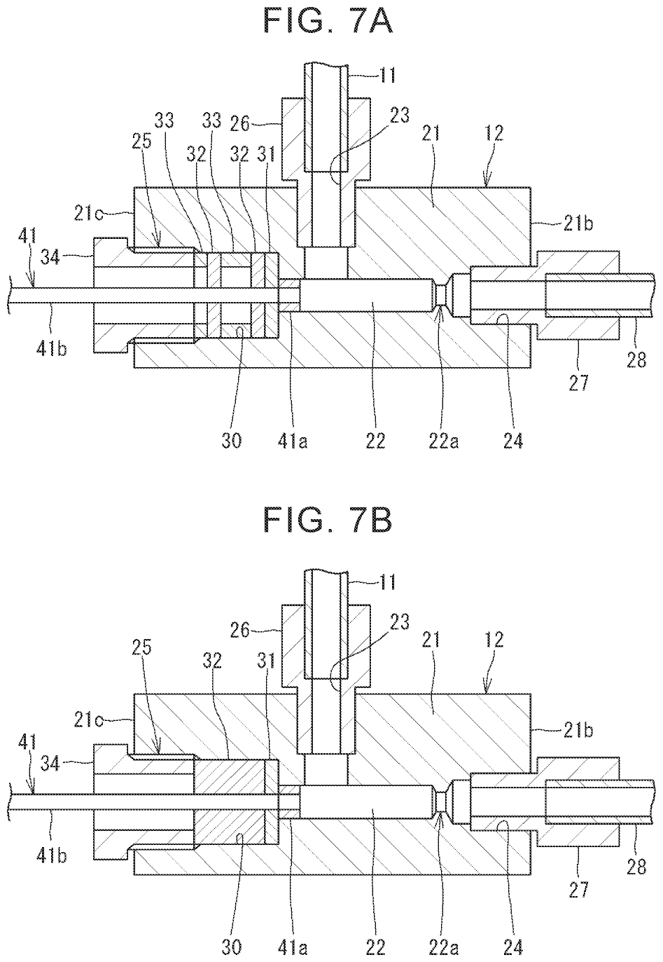

FIGS. 7A, 7B are sectional views of modified examples of the reservoir unit 12 in the property measurement device 10. In the reservoir unit 12 shown in FIG. 7A, the two support rings 32 and the spacer 33 are provided in the piston support portion 25, and the piston 41 is supported at two points by the two support rings 32. Thus, the piston 41 is further stably supported, and reciprocating motion of the piston 41 in the reservoir chamber 22 is further stabilized.

The reservoir unit 12 shown in FIG. 7B is not provided with the spacer. The support ring 32 is provided in an entire area between the seal member 31 and the fixing member 34, and the support ring 32 extends along the center line O1. Thus, the piston 41 is further stably supported in the larger area, and the reciprocating motion of the piston 41 in the reservoir chamber 22 is further stabilized.

The disclosure is not limited to the above-described embodiment and modified examples, and various changes and modifications may be made within the scope of the disclosure. The disclosure is not limited to the rolling bearing that supports the main shaft of the wind power generation system and can measure the property of the grease that is used in any of the rolling bearings used for various purposes. In addition, the disclosure is not limited to the self-aligning roller bearing described in the above embodiment and can measure the property of the grease used in any of the various rolling bearings.

Furthermore, the property of the grease measured by the property measurement device is not limited to the consistency and may be another property as long as the property is correlated with the flow resistance of the grease.

What is claimed is:

1. A grease property measurement device comprising:
a flow pipe having a first end that communicates with an inside of a rolling bearing such that grease discharged from the rolling bearing flows through the flow pipe;
a reservoir unit including an inlet port to which a second end of the flow pipe is connected such that the grease is introduced from the flow pipe through the inlet port, a reservoir chamber which stores the grease introduced through the inlet port, and a discharge port through which the grease is discharged from the reservoir chamber;
an extrusion mechanism configured to push the grease in the reservoir chamber so as to discharge the grease through the discharge port; and
a measuring unit configured to measure flow resistance at a time when the extrusion mechanism pushes the grease in the reservoir chamber.

2. The grease property measurement device according to claim 1, wherein the extrusion mechanism includes a piston provided to reciprocate in a first direction to push the grease in the reservoir chamber and in a second direction opposite to the first direction; and a drive unit configured to cause the piston to reciprocate.

3. The grease property measurement device according to claim 2, wherein the measuring unit includes a pressure sensor that is provided between the piston and the drive unit, and the pressure sensor detects a pressure applied to the piston from the drive unit.

4. The grease property measurement device according to claim 3, wherein:
the extrusion mechanism further includes a coupling unit that couples the piston and the drive unit; and
the coupling unit is configured to cause the pressure sensor to contact both of the drive unit and the piston when the piston moves in the first direction, and to cause the pressure sensor to move away from one of the drive unit and the piston when the piston moves in the second direction.

5. The grease property measurement device according to claim 1 further comprising
a check valve configured to permit a flow of the grease in a direction from the flow pipe toward the inlet port and to prevent a flow of the grease in a direction opposite to the direction from the flow pipe toward the inlet port.

6. The grease property measurement device according to claim 3, wherein the pressure sensor is attached to one of the piston and the drive unit.

7. A grease property measurement method comprising:
introducing grease in a rolling bearing into a reservoir chamber through a flow pipe;
pushing the grease in the reservoir chamber so as to discharge the grease from the reservoir chamber; and
measuring flow resistance at a time when the grease is pushed and discharged from the reservoir chamber.
